(19) United States
(12) Plant Patent Application Publication
(10) Pub. No.: US 2003/0145362 P1
(43) Pub. Date: Jul. 31, 2003

(54) CALIBRACHOA PLANT NAMED 'KAKEGAWA S44'

(76) Inventor: Masao Bessho, Kakegawa City (JP)

Correspondence Address: JONDLE & ASSOCIATES P.C., 9085 EAST MINERAL CIRCLE, SUITE 200, CENTENNIAL, CO, 80112, US

(21) Appl. No.: 10/058,775

(22) Filed: Jan. 30, 2002

(51) Int. Cl. A01H 5/00

(52) U.S. Cl. PLT/263

(57) ABSTRACT

‘Kakegawa S44’ is a new variety of Calibrachoa. This new variety has lavender colored flowers and dark green foliage.

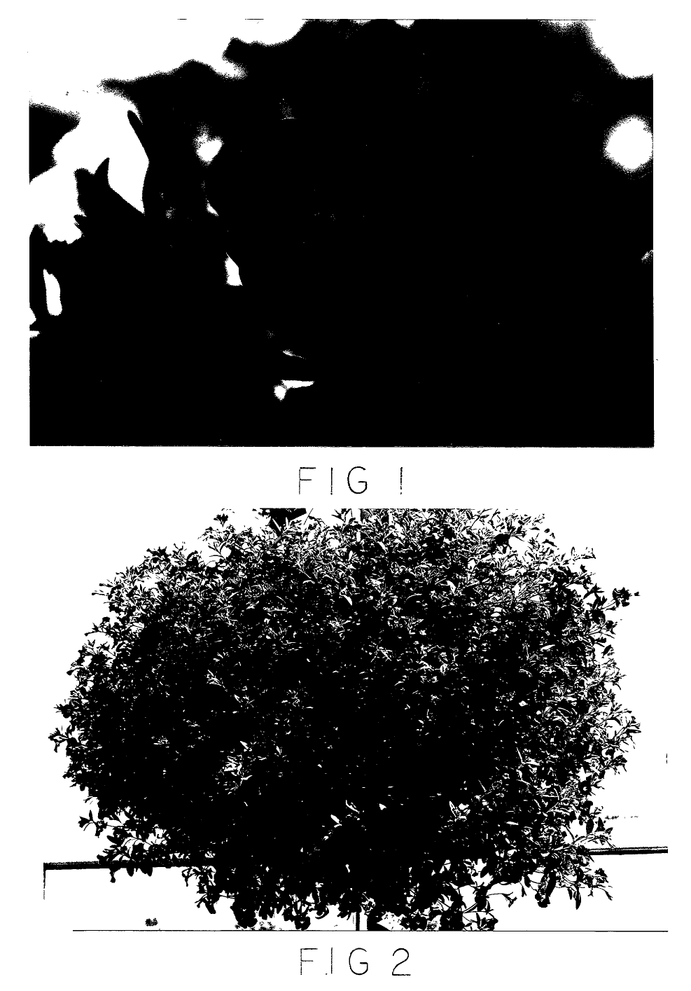

### BACKGROUND OF THE INVENTION

‘Kakegawa S44’ originated from a hybridization made in spring 1995 at Kakegawa, Japan. The female parent was the commercial variety Liricashower Rose (U.S. Plant Pat. No. 9,884). The male parent was a breeding line obtained from a commercial market in Brazil and was known only as line C-13CDE (Not patented). Seed from this cross was sown in spring 1996 and three F₁ plants were selected with rose colored flowers. The three selections were intercrossed and the resulting seed bulked together to produce F₂ seed. In spring 1997 the F₂ seed was sown and one plant, designated as line SK7-1169, was selected. This line was vegetatively propagated in fall 1997 and all progeny were stable and uniform. In spring 1998 the line was vegetatively propagated and cuttings were delivered to Salinas, Calif. The new variety was vegetatively propagated again in spring 1999. In these three vegetative generations the line was evaluated and determined that the traits were firmly fixed and stable. No inherent variation or off-types have been identified.

### DESCRIPTION OF THE GENUS CALIBRACHOA LLAVE & LEX

The genus Petunia was originally established in 1803 by A. L. Jussieu, who described both P. parviflora and P. nyctaginifloa as type species. Using a non-horticultural system that selected the first mentioned species as the type species (lectotype), N. L. Britton and H. A. Brown declared P. parviflora as the type species for Petunia in 1913.

During the 1980's and 1990, H. J. Wijsman published a series of articles regarding the ancestry of P. hybrida, the Garden Petunia, and the inter-relationship of several species classified as Petunia. These studies discovered that P. hybrida and its ancestrial species, P. nyctaginiflora (=P. axillaris) and P. violacea (=P. integrifolia), possessed 14 pairs of chromosomes while several other species, including P. parviflora, possessed 18 pairs of chromosomes. Since P. parviflora was the lectotype species for the Petunia genus, Wijsman and J. H. de Jong proposed transferring the 14 chromosome species to the genus Stimoryne. Horticulturists opposed reclassifying the Garden Petunia and in 1986, Wijsman proposed the alternative of making P. nyctaginiflora the lectotype species for Petunia and transferring the 18 chromosome species to another genus. The I. N. G. Committee adopted this proposal. By 1990 Wijsman had transferred several species, including P. parviflora (=C. parviflora) to Calibrachoa, originally established by Llave and Lexarza in 1825. Calibrachoa parviflora (=C. mexicana Llave & Lexarza) is now the type species for the genus Calibrachoa.

Classification of the current Petunia and Calibrachoa species is still in progress. New species are also being identified. Consequently a proper description has not been written for the Calibrachoa genus. Calibrachoa can, however, be distinguished from Petunia based on the higher chromosome number, chromosome morphology, plant branching habit and type of flower bud aestivation. Whereas Petunia species bear a flower peduncle and one new stem from a node, Calibrachoa bear a flower peduncle and three stems. Petunia species have a cochlear corolla bud, a single outermost petal covers the other four, radially folded and terminally contorted petals. Calibrachoa flower buds are flat with all five petals linearly folded and the two lower petals forming a cover around the three other petals and fused together.

### ENVIRONMENTAL CONDITIONS FOR PLANT GROWTH

Vegetative cuttings were propagated in five to six weeks. Cuttings were stuck into plastic cell trays having 98 cells, and containing a peat moss-based growing medium. The base of the cuttings were dipped for 1 to 2 second s in a 1:9 solution of Dip 'N Grow (1 part solution to 9 parts water) root inducing solution immediately prior to sticking into the cell trays. The cuttings were misted with water from overhead for 10 seconds every 30 minutes until sufficient roots were formed.

Rooted cuttings were transplanted and grown in 20 cm diameter plastic pots in a glass greenhouse located in Salinas, Calif. Pots contained a peat moss-based growing medium. Soluble fertilizer containing 20% nitrogen, 10% phosphorus and 20% potassium was applied once a day or every other day by overhead irrigation. Pots were top-dressed with a dry, slow release fertilizer containing 20% nitrogen, 10% phosphorus and 18% potassium. The typical average air temperature was 24C.

### DETAILED DESCRIPTION OF THE NEW PLANT

The following traits and characteristics describe the new variety.

Classification:

Family.—Solanaceae.

Species.—Calibrachoa spp.

Common names.—Petunia.

Parentage:

Female parent.—Liricashower Rose (U.S. Plant Pat. No. 9,884).

Male parent.—breeding line C-13CDE (Not Patented).

Growth:

Habit.—decumbent, mounding in center.

Height.—23.0 cm.

Spread.—115.0 cm when grown in a 41 cm hanging basket or pot, and using five 20 cm potted plants in one hanging basket.

Life cycle.—annual.

Time to produce a rooted cutting.—3 weeks.

Time to bloom from propagation.—10 weeks.

Flowering season.—spring and summer.

Flowering requirements.—air temperatures of 13-15C during the night and 13-20C during the day; day neutral light requirement.

Resistance/susceptibility.—excellent resistance to rain, heat and drought; will not tolerate temperatures below 10C.

Form.—branching, dense.

Stems:

Stem color.—R.H.S. 144B (yellow-green).

Anthocyanin color.—R.H.S. 77A (purple).

Pubescence.—heavy.

Pubescence color.—R.H.S. 155D (white).

Pubescence shape.—pointed.

Stem description.—round, ancipital.

Stem diameter.—2.0 mm.

Internode length.—1.5 to 2.5 cm.

Leaves:

Leaf tip.—mucronate.

Leaf arrangement.—alternate.

Leaf base.—decurrent.

Leaf color.—upper surface: R.H.S. 137A (green); lower surface: R.H.S. 138B (green).

Leaf edge.—entire.

Leaf fragrance.—absent.

Leaf surface.—rough.

Leaf surface pubescence.—slightly.

Petiole color.—R.H.S. 138B (green).

Petiole length.—2.0 mm.

Leaf length.—3.0 cm.

Leaf shape.—spatulate.

Leaf width.—1.0 cm.

Flowers:

Calyx.—5 sepals; 8.0×2.0 mm (l×w); free.

Corolla.—5 petals, fused.

Flower diameter.—3.0 cm.

Bud color.—R.H.S. N144D (yellow-green).

Bud shape.—ovate.

Bud surface.—pubescent.

Duration of flower life.—5 days.

Flowering habit.—indeterminate.

Ovary.—superior.

Placenta arrangement.—central.

Peduncle size.—2.5 cm×1.0 mm (l×w); slightly smooth, short pubescence.

Inflorescence type.—solitary.

Stamens.—5 stamens, three short and two long, free.

Stamens color.—R.H.S 144D (yellow-green).

Stigma color.—R.H.S. 134A (green).

Style color.—R.H.S. 144D (yellow-green)

Petal size.—3.0×1.5 cm (l×w).

Petal pubescence.—glabrous.

Petal color.—upper surface: R.H.S. N81B (purple-violet); lower surface:

R.H.S. N81D (purple-violet); corolla tube: inner surface R.H.S. 13A (yellow); outer surface R.H.S. 3C (yellow) with R.H.S. N77A (purple) veins.

Pollen color.—R.H.S. 1A (green-yellow).

Produces seed.—yes.

### COMPARISON WITH MOST SIMILAR VARIETY

‘Kakegawa S44’ is most similar to the variety ‘Colorburst Violet’ (U.S. Plant Pat. No. 12,086). The two varieties are similar in foliage, plant habit and flower size. The two plants are different because ‘Colorburst Violet’ has purple flowers while ‘Kakegawa S44’ has lavender flowers.

What is claimed is:

1. A new and distinct cultivar of Calibrachoa as shown and described herein.
